(19) United States
(12) Patent Application Publication
(10) Pub. No.: US 2026/0175038 A1
(43) Pub. Date: Jun. 25, 2026

(54) Systems And Methods For Indicating Neural Responses

(71) Applicant: Cochlear Limited, Macquarie University, NSW (AU)

(72) Inventor: Andrew Wing Fu Lang, Sydney (AU)

(73) Assignee: Cochlear Limited, Macquarie University, NSW (AU)

(21) Appl. No.: 19/124,862

(22) PCT Filed: Oct. 23, 2023
(86) PCT No.: PCT/IB2023/060696

§ 371 Date: Apr. 26, 2025

Related U.S. Application Data
(60) Provisional application No. 63/421,339, filed on Nov. 1, 2022.

(51) Int. Cl. A61N 1/372; A61B 5/24; A61N 1/05; G06N 3/08

(52) U.S. Cl. CPC A61N 1/37247; A61B 5/24; A61N 1/0541; G06N 3/08

(57) ABSTRACT

A computing system includes a processing unit that implements an artificial neural network. The artificial neural network generates an output at a single output node that indicates whether a measurement performed after a stimulus to a neural region of an individual includes a neural response. A method includes receiving, at an artificial neural network in a computing system, values indicative of a measurement performed after a stimulus provided to a neural region of an individual, and generating an output that indicates whether the measurement includes a neural response at a single output node of the artificial neural network.

### CROSS REFERENCE TO RELATED APPLICATION

This patent application claims priority to U.S. provisional patent application 63/421,339, filed Nov. 1, 2022, which is incorporated by reference herein in its entirety.

### FIELD OF THE DISCLOSURE

The present disclosure relates to systems and methods for indicating neural responses in individuals in computing systems.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### BRIEF SUMMARY

According to a first embodiment disclosed herein, a computing system includes at least one processing unit that implements an artificial neural network, wherein the artificial neural network generates an output at a single output node that indicates whether a measurement performed after a stimulus to a neural region of an individual includes a neural response.

According to a second embodiment disclosed herein, a method comprises receiving, at an artificial neural network in a computing system, values indicative of a measurement performed after a stimulus provided to a neural region of an individual; and generating an output that indicates whether the measurement comprises a neural response at a single output node of the artificial neural network.

According to a third embodiment disclosed herein, a non-transitory computer-readable storage medium comprising computer-readable instructions stored thereon for causing a computing system to: receive, at input nodes of an artificial neural network in the computing system, pixels from an image of a trace of a measurement performed after a stimulus to an auditory nerve of an individual; and generate an indication of whether the measurement comprises a neural response based on the pixels using the artificial neural network.

According to a fourth embodiment disclosed herein, a method comprising: sampling a signal indicative of a measurement performed after a stimulus to an auditory nerve of an individual to generate a sampled signal; performing a Fourier transform of the sampled signal to extract frequency components of the sampled signal; receiving the frequency components of the sampled signal at input nodes of an artificial neural network in a computing system; and generating an output indicating whether the measurement comprises a neural response using the artificial neural network.

### BRIEF DESCRIPTION OF THE DRAWINGS

FIG. 1A depicts a schematic diagram of an exemplary cochlear implant system that can be configured to implement aspects of the techniques presented herein, according to some exemplary embodiments.

FIG. 1B depicts a block diagram of the cochlear implant system of FIG. 1A.

FIG. 2 depicts a diagram illustrating an example of an artificial neural network (ANN) that can be used to determine if a measurement performed after a stimulus to a neural region of a recipient includes a neural response to the stimulus.

FIG. 3 depicts a flow chart that illustrates examples of operations that can be performed to train an artificial neural network (ANN) to determine whether a measurement of neural activity performed after a stimulus to a neural region of a recipient includes a neural response.

FIG. 4 depicts a flow chart that illustrates examples of operations that can be performed to determine if a measurement of neural activity performed after applying a stimulus to a neural region of a recipient includes a neural response to the stimulus using an artificial neural network (ANN) that has been trained according to the operations of FIG. 3.

FIG. 5 depicts a flow chart that illustrates examples of operations that can be performed to determine a stimulus level that evokes a neural response from an auditory nerve of a recipient within a search range of stimuli levels.

FIG. 6 depicts a flow chart that illustrates examples of operations that can be performed to determine if a measurement performed after applying a stimulus to a neural region of a recipient includes a neural response by providing pixels of a trace of the measurement to an artificial neural network (ANN).

FIG. 7 depicts a flow chart that illustrates examples of operations that can be performed to determine if a measurement performed after applying a stimulus to a neural region of a recipient includes a neural response by providing frequency components of a sample of the measurement to an artificial neural network (ANN).

FIG. 8 depicts a flow chart that illustrates examples of operations that can be performed to determine if a measurement performed after applying a stimulus to a neural region of a recipient includes a neural response by providing samples of a signal indicative of the measurement to an artificial neural network (ANN).

FIG. 9 illustrates an example of a suitable computing system that can perform any of the operations or functions disclosed herein.

### DETAILED DESCRIPTION

Hearing loss in an individual may have many different causes. Sensorineural hearing loss is the cause of deafness in many people. Sensorineural hearing loss is caused by the absence or destruction of the hair cells in the cochlea that transduce acoustic signals into nerve impulses. Individuals suffering from sensorineural hearing loss are unable to derive suitable benefit from conventional hearing aids due to the damage to, or absence, of the mechanism for naturally generating nerve impulses from sound. Cochlear implant systems are a type of auditory prosthesis that has been developed to potentially address sensorineural hearing loss. Cochlear implant systems bypass the hair cells in the cochlea, directly delivering electrical stimulation to the auditory nerve fibers via an implanted electrode assembly. The electrical stimulation enables the brain to perceive a hearing sensation resembling the natural hearing sensation normally delivered to the auditory nerve fibers.

Cochlear implant systems have traditionally included an external speech processor unit worn on the body of the recipient and a receiver/stimulator unit implanted in the recipient. The external speech processor unit detects external sounds and converts the detected external sounds into a coded signal through a speech processing strategy. The coded signal is sent to the implanted receiver/stimulator unit via a transcutaneous link. The receiver/stimulator unit processes the coded signal to generate a series of stimulation sequences that are then applied directly to the auditory nerve via a series-arrangement or an array of electrodes positioned within the cochlea.

The external speech processor unit and the implanted receiver/stimulator unit can be combined to produce a totally implantable cochlear implant system capable of operating, at least for a period of time, without the need for an external device. In such an implant, a microphone is implanted within the body of the recipient, for example, in the ear canal or within the stimulator unit. Detected sound is directly processed by a speech processor within the stimulator unit, with the subsequent stimulation signals delivered without the need for any transcutaneous transmission of signals.

Data is obtained from the components of a cochlear implant system to enable detection and confirmation of normal operation of the cochlear implant system The data can also be obtained from a cochlear implant system to allow stimulation parameters to be optimized to suit the needs of different recipients, including data relating to the response of the auditory nerve to stimulation. A cochlear implant system typically has the capability to communicate with an external device, for example, to receive program upgrades, to perform implant interrogation, and to read and/or alter the operating parameters of the cochlear implant system.

Determining the response of an auditory nerve to stimulation has been addressed with limited success in conventional systems. Typically, following the surgical implantation of an implantable component of a cochlear implant system, the cochlear implant system is fitted or customized to conform to specific recipient needs. The customization procedure can involve the collection and determination of patient-specific parameters, such as threshold levels (T levels) and maximum comfort levels (C levels) for each stimulation channel in the cochlear implant system. In previously known systems, the customization procedure is performed manually by applying stimulation pulses for each stimulation channel and receiving an indication from the recipient as to the level and comfort of the resulting sound. For cochlear implant systems having a large number of channels for stimulation, the customization procedure is time consuming and subjective, because the customization procedure relies heavily on the recipient's subjective impression of the stimulation rather than an objective measurement.

Performing the customization procedure manually is further limited for children and prelingually or congenitally deaf patients who are unable to supply an accurate impression of the resultant hearing sensation. For these recipients, fitting of the cochlear implant system may be sub-optimal. An incorrectly-fitted cochlear implant system may result in the recipient not receiving optimum benefit from the cochlear implant system. For example, an incorrectly-fitted cochlear implant system in a child may directly hamper the speech and hearing development of the child. Therefore, there is a need to obtain objective measurements of patient-specific data, such as minimum threshold levels (T levels) and maximum comfort levels (C levels) for stimulation channels in a cochlear implant system, particularly in situations when an accurate subjective measurement is not possible.

One technique for interrogating the performance of a cochlear implant system and making objective measurements of patient-specific data, such as T and C levels, is to directly measure the response of the auditory nerve to an electrical stimulus. The direct measurements of neural responses, commonly referred to as Electrically-evoked Compound Action Potentials (ECAPs) in the context of cochlear implant systems, provide objective measurements of the responses of auditory nerves to electrical stimuli. Following electrical stimulation, the neural response is caused by the superposition of neural responses at the outside of the axon membranes. Measurements from within the cochlea can be taken in response to various stimulations. The measurements are taken to determine whether a neural response has occurred. The measurements are objective measurements of neural activity. Generally, neural activity of the auditory nerve resulting from a stimulus presented at one electrode in an implantable component of a cochlear implant system is measured at another electrode in the implantable component (e.g., at a neighboring electrode). The measurements are typically transmitted to an externally-located system.

Cochlear implant systems typically have the ability to generate stimulation using one electrode and to measure neural activity after the stimulation at an adjacent electrode. When the stimulus is large enough to cause an Electrically-evoked Compound Action Potential (ECAP) in an auditory nerve, the waveform of the measured potential takes on a distinctive shape that can be seen by the human eye. The minimum stimulus amplitude required to generate an ECAP may be referred to as the threshold of the neural response. The conventional technique for determining a neural response of a recipient of a cochlear implant system is a manual process that involves providing electrical stimulus to an auditory nerve of the recipient at increasing amplitudes using electrodes in the implantable component and then analyzing measurements taken after the electrical stimulus for ECAPs. This manual process for determining neural responses is time consuming and subject to the variation of human expertise and experience. Therefore, it would be desirable to provide an automated system for detecting neural responses. It would also be desirable to provide a simplified and efficient system that can be used in a clinic for a large patient base.

According to some embodiments disclosed herein, systems and methods are provided for receiving at an artificial neural network (ANN) in a computing system (e.g., in an electrophysiological response measurement system) values indicative of a measurement performed after a stimulus provided to a neural region of an individual, and generating an output that indicates whether the measurement includes a neural response at a single output node of the artificial neural network. The values may, for example, include values from a signal indicative of a measurement of neural activity taken after an electrical stimulus is delivered by an electrode in an implant system (such as cochlear implant system) to the auditory nerve of the recipient of the implant system. According to other embodiments disclosed herein, systems and methods are provided for receiving at an input layer of an artificial neural network (ANN) pixels, samples, or frequency components of a signal indicative of a measurement of neural activity performed after a stimulus to a neural region of an individual; and generate an indication of whether the measurement comprises a neural response using the ANN. Advantageously, the present technology can provide a binary output indicating whether a neural response has been evoked, and as a result, the present technology can be used more universally across a range of different kinds of patients, while also streamlining the clinical process. Further details of these embodiments and other embodiments are disclosed below.

Merely for ease of description, the techniques presented herein are primarily described herein with reference to an illustrative medical device, namely a cochlear implant system. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant system, corresponds to a disclosure of another embodiment of using such teaching with another hearing prostheses, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system and/or method that is used to, or results in, stimulation of the auditory nerve corresponds to a disclosure of an analogous stimulation of the optic nerve utilizing analogous components, methods, and systems.

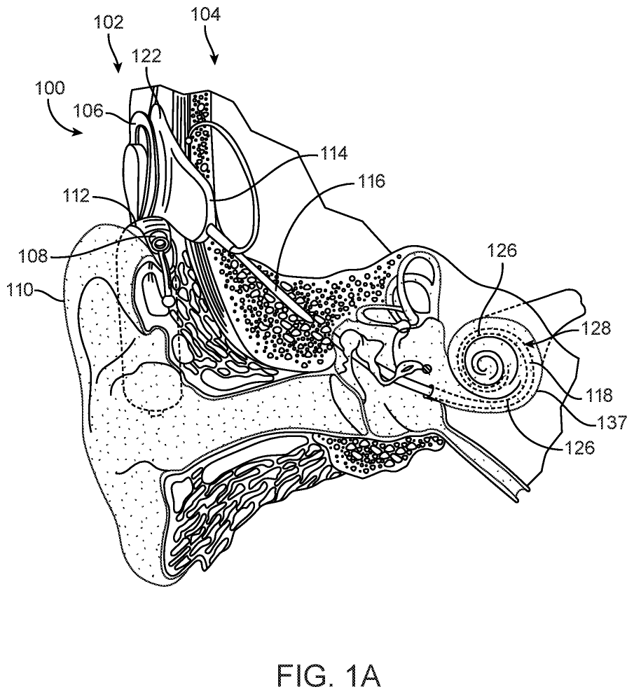

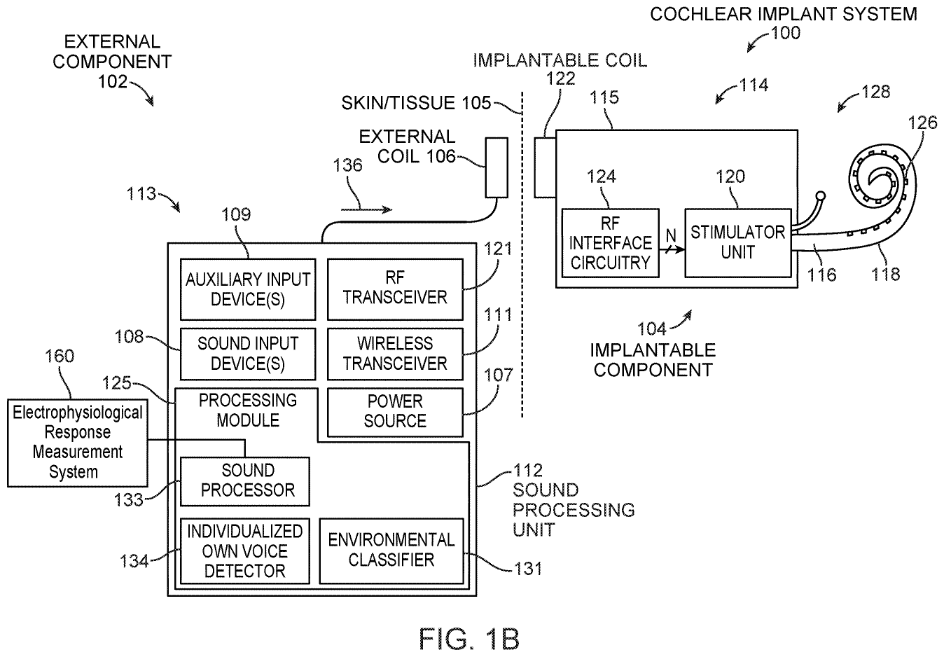

FIG. 1A is a schematic diagram of an exemplary cochlear implant system 100 configured to implement aspects of the techniques presented herein. FIG. 1B is a block diagram of the cochlear implant system 100 of FIG. 1A. For ease of illustration, FIGS. 1A and 1B are described together herein. The cochlear implant system 100 includes an external component 102 and an internal/implantable component 104. The external component 102 is directly or indirectly attached to the body of the recipient and typically comprises an external coil 106 and, generally, a magnet (not shown in FIGS. 1A-1B) fixed relative to the external coil 106. The external component 102 also comprises one or more input elements/devices 113 (shown in FIG. 1B) for receiving input signals at a sound processing unit 112. In this example, the one or more input devices 113 include sound input devices 108 (e.g., microphones positioned by auricle 110 of the recipient, telecoils, etc.) configured to capture/receive input signals, one or more auxiliary input devices 109 (e.g., audio ports, such as a Direct Audio Input (DAI), data ports, such as a Universal Serial Bus (USB) port, cable port, etc.), and a wireless transmitter/receiver (transceiver) 111, each located in, on, or near the sound processing unit 112.

The sound processing unit 112 also includes, for example, at least one power source 107, a radio-frequency (RF) transceiver 121, and a processing module 125. The processing module 125 includes a number of elements, including an environmental classifier 131, a sound processor 133, and an individualized own voice detector 134. Each of the environmental classifier 131, the sound processor 133, and the individualized own voice detector 134 can be formed by one or more processors (e.g., one or more Digital Signal Processors (DSPs), one or more processing cores, etc.), firmware, software, etc. arranged to perform operations described herein. That is, the environmental classifier 131, the sound processor 133, and the individualized own voice detector 134 can each be implemented as firmware elements, partially or fully implemented with digital logic gates in one or more application-specific integrated circuits (ASICs), partially or fully in software, etc.

In the examples of FIGS. 1A and 1, the sound processing unit 112 is a behind-the-ear (BTE) sound processing unit configured to be attached to, and worn adjacent to, the recipient's ear. However, it is to be appreciated that sound processing unit 112 can have other arrangements, such as an off the ear (OTE) processing unit (e.g., a component having a generally cylindrical shape and that is configured to be magnetically coupled to the recipient's head), etc., a mini or micro-BTE unit, an in-the-canal unit that is configured to be located in the recipient's ear canal, a body-worn sound processing unit, etc.

In the exemplary embodiment of FIGS. 1A and 1B, the implantable component 104 includes an implant body (main module) 114, a lead region 116, and an intra-cochlear stimulating assembly 118, all configured to be implanted under the skin/tissue (tissue) 105 of the recipient. The implant body 114 generally includes a hermetically-sealed housing 115 in which RF interface circuitry 124 and a stimulator unit 120 are disposed. The implant body 114 also includes an internal/implantable coil 122 that is generally external to the housing 115, but that is connected to the RF interface circuitry 124 via a hermetic feedthrough (not shown in FIG. 1).

Stimulating assembly 118 is configured to be at least partially implanted in the recipient's cochlea 137. Stimulating assembly 118 includes a plurality of longitudinally spaced intra-cochlear electrical stimulating contacts (e.g., electrodes) 126 that collectively form a contact or electrode array 128 for delivery of electrical stimulation (current) to the recipient's cochlea. Stimulating assembly 118 extends through an opening in the recipient's cochlea (e.g., cochleostomy, the round window, etc.) and has a proximal end connected to stimulator unit 120 via lead region 116 and a hermetic feedthrough (not shown in FIG. 1). Lead region 116 includes a plurality of conductors (wires) that electrically couple the stimulating contacts 126 to the stimulator unit 120.

As noted, the cochlear implant system 100 includes the external coil 106 and the implantable coil 122. The coils 106 and 122 are typically wire antenna coils each comprised of multiple turns of electrically insulated single-strand or multi-strand wire. Generally, a magnet is fixed in position relative to each of the external coil 106 and the implantable coil 122. In some embodiments, the external component 102 and/or the implantable component 104 can include magnet assemblies that each have more than one magnetic component. The magnets fixed relative to the external coil 106 and the implantable coil 122 facilitate the operational alignment of the external coil with the implantable coil. This operational alignment of the coils 106 and 122 enables the external component 102 to transmit data, as well as possibly power, to the implantable component 104 via a closely-coupled wireless link formed between the external coil 106 and the implantable coil 122. In certain examples, the closely-coupled wireless link is a radio frequency (RF) link. However, various other types of energy transfer, such as infrared (IR), electromagnetic, capacitive and inductive transfer, can be used to transfer the power and/or data from an external component to an implantable component and, as such, FIG. 1B illustrates only one exemplary arrangement.

As noted above, sound processing unit 112 includes the processing module 125. The processing module 125 is configured to convert input audio signals into stimulation control signals 136 for use in stimulating a first ear of a recipient (i.e., the processing module 125 is configured to perform sound processing on input audio signals received at the sound processing unit 112). Stated differently, the sound processor 133 (e.g., one or more processing elements implementing firmware, software, etc.) is configured to convert the captured input audio signals into stimulation control signals 136 that represent electrical stimulation for delivery to the recipient. The input audio signals that are processed and converted into stimulation control signals 136 can be audio signals received via the sound input devices 108, signals received via the auxiliary input devices 109, and/or signals received via the wireless transceiver 111.

In the embodiment of FIG. 1B, the stimulation control signals 136 are provided to the RF transceiver 121, which transcutaneously transfers the stimulation control signals 136 (e.g., in an encoded manner) to the implantable component 104 via external coil 106 and implantable coil 122. The stimulation control signals 136 are received at the RF interface circuitry 124 via implantable coil 122 and provided to the stimulator unit 120 (e.g., as an N number of signals). The stimulator unit 120 is configured to utilize the stimulation control signals 136 to generate electrical stimulation signals (e.g., current signals) for delivery to the recipient's cochlea via one or more stimulating contacts 126 (e.g., electrode) in array 128. In this way, cochlear implant system 100 electrically stimulates the recipient's auditory nerve cells, bypassing absent or defective hair cells that normally transduce acoustic vibrations into neural activity, in a manner that causes the recipient to perceive one or more components of the input audio signals.

FIG. 1B also illustrates an electrophysiological response measurement system 160 that is communicably coupled to the sound processor 133 via a connection (e.g., a cable). The electrophysiological response measurement system 160 is, in some embodiments, a processor-based system such as a personal computer, server, workstation or the like, having one or more processors that execute software programs to perform the techniques disclosed herein. For example, system 160 can generate a signal that is used by the cochlear implant system 100 as a stimulus to stimulate the auditory nerve of the recipient via one or more stimulating contacts 126, receive a measurement of neural activity in response to the stimulus from the cochlear implant system 100, and generate an indication of whether the measurement of neural activity includes a neural response or does not include a neural response of the auditory nerve of the recipient.

According to some embodiments disclosed herein, electrophysiological response measurement system 160 includes a computer system that implements an artificial neural network (ANN). The ANN receives a representation (e.g., a visual or frequency based representation) of a measurement of neural activity performed after a stimulus provided to an auditory nerve of a recipient and classifies the representation as including a neural response or not including a neural response to the stimulus. The ANN can, for example, determine that the measurement does not include a neural response if the measurement includes only noise. The ANN can be incorporated into a search algorithm that generates signals provided to the auditory nerve of the recipient as stimuli at varying stimuli levels, receives measurements of neural activity in response to the stimuli, and determines whether the measurements include neural responses.

The artificial neural network (ANN) can include an input layer, one or more hidden layers, and an output layer. The input layer of the ANN includes input nodes. The number of input nodes in the input layer of the ANN can be selected based on the data that is provided to the input layer, as described in further detail below. The ANN can have any number of one or more hidden layers. The number of hidden layers in the ANN can be selected according to user preference. Each of the hidden layers has one or more hidden nodes. The output layer of the ANN can include only a single output node.

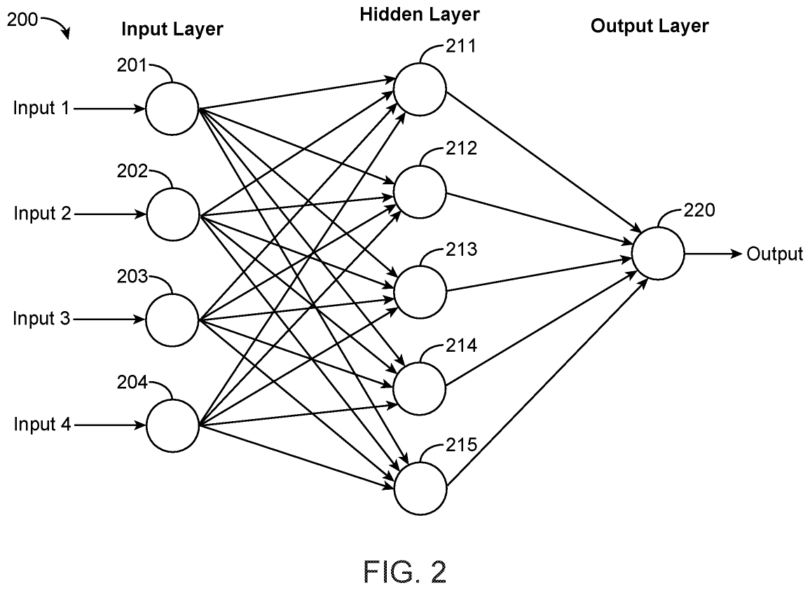

FIG. 2 depicts a diagram illustrating an example of an artificial neural network (ANN) that can be used to determine if a measurement performed after a stimulus to a neural region of a recipient includes a neural response to the stimulus. ANN 200 includes an input layer, a hidden layer, and an output layer. Although only a single hidden layer is shown in FIG. 2 as an example, it should be understood that ANNs used to implement the techniques disclosed herein can include any number of hidden layers. In the example of ANN 200, the input layer includes 4 input nodes 201-204, and the hidden layer includes 5 hidden nodes 211-215. The number of nodes shown in FIG. 2 in the input and hidden layers are provided merely as examples. It should be understood that each of the input layer and the hidden layer in an ANN used to implement techniques disclosed herein can have any number of nodes (e.g., hundreds or thousands of nodes). The output layer of ANN 200 has only a single output node 220. Inputs 1-4 are values provided to input nodes 201-204, respectively. The input provided to each of the hidden nodes (e.g., hidden nodes 211-215) and to the output node 220 is a weighted sum s of the outputs of the nodes in the previous layer, as shown in equation (1) below, where w₀ is a constant, and w₁, w₂, . . . are weights applied to the outputs x₁, x₂, . . . of the nodes in the previous layer.

$$s = {w}_{1} {x}_{1} + {w}_{2} {x}_{2} + {w}_{0} \qquad ( 1 )$$

As an example, the input to hidden node 211 is the weighted sum s₂₁₁ of the outputs of the nodes 201-204, i.e., s₂₁₁=w₁x₁+w₂x₂+w₃x₃+w₄x₄, where x₁, x₂, x₃, x₄ are the outputs of nodes 201-204, which equal Inputs 1-4, respectively. The output of each of the hidden nodes 211-215 and of the output node 220 is a transfer function ƒ(s). The transfer function ƒ(s) can be, for example, a differentiable function, such as a sigmoid or hyperbolic tangent function (i.e., tanh), as shown in equation (2) below. In equation (2), s is the output of equation (1), and e is the mathematical constant known as Euler's number.

$$output = f ( s ) = \frac{1}{1 + {e}^{- s}} \qquad ( 2 )$$

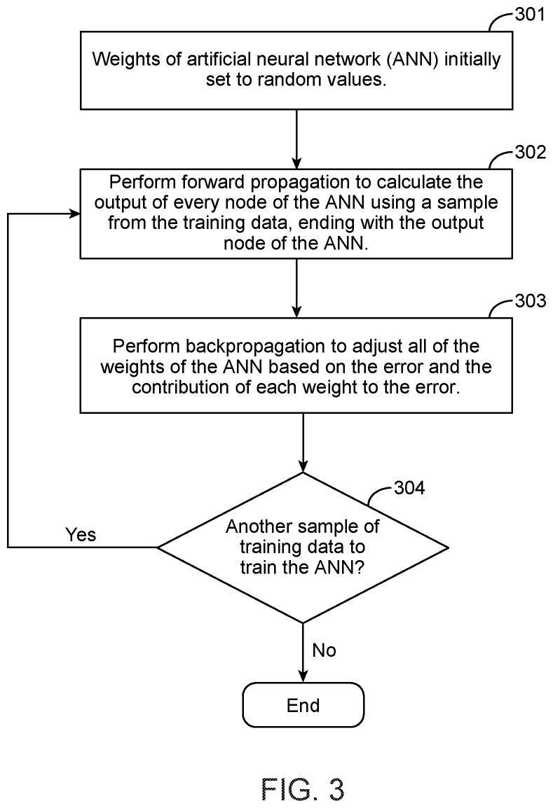

FIG. 3 depicts a flow chart that illustrates examples of operations that can be performed to train an artificial neural network (ANN) to determine whether a measurement of neural activity performed after a stimulus to a neural region of a recipient includes a neural response. The operations of FIG. 3 are performed by a computer system. ANN 200 is an example of an ANN that can be trained using the operations of FIG. 3. In addition, the operations of FIG. 3 can also be used to train other ANNs to determine whether a measurement of neural activity performed after a stimulus to a neural region of a recipient includes a neural response. In operation 301, the weights of the ANN are initially set to random values. In the example of FIG. 2, the weights w₁, w₂, . . . of ANN 200 that are applied to the outputs of the nodes 201-204 in the input layer and to the outputs of the nodes 211-215 in the hidden layer are initially set to random values in operation 301.

The operations of FIG. 3 can be used to train an ANN using training data. The training data includes measurements of neural activity evoked in a neural region of a recipient (e.g., the auditory nerve) in response to stimuli provided to the neural region of the recipient (e.g., of a cochlear implant system) and labels or classifications that indicate whether each of the measurements includes a neural response or does not include a neural response. The labels or classifications can be, for example, evaluations by a human expert that indicate whether each of the measurements includes a neural response. As examples, the stimuli can be electrical stimuli to the auditory nerve that are generated by one or more electrodes (e.g., stimulating contacts 126) in a cochlear implant system in response to signals generated by an electrophysiological response measurement system, and the measurements can be sensed by one or more electrodes in the cochlear implant system. The measurements can be provided to the electrophysiological response measurement system. The measurements can be, for example, processed by the electrophysiological response measurement system (e.g., to generate traces on a display screen). Using training data having a large number of labeled or classified measurements helps to train the ANN to subsequently determine whether unlabeled or unclassified measurements include neural responses. In general, the ANN can more accurately determine if the measurements include neural responses if the ANN has been trained with training data having a larger number of measurements.

In operation 302, forward propagation is performed on the ANN to calculate the output of every node of the ANN using a sample from the training data discussed above, ending with the output node of the ANN. The sample includes values that are provided to the input nodes in the input layer of the ANN in operation 302. Each sample used in operation 302 can, for example, include one or more measurements of neural activity evoked in a neural region of a recipient (e.g., the auditory nerve) in response to stimuli provided to the neural region of the recipient, as discussed above. As a more specific example, each sample used in operation 302 can include values from a measurement performed by a cochlear implant system. The output of operation 302 is a value generated by the output node of the ANN that indicates if the sample represents a neural response or does not represent a neural response.

The output of operation 302 is then compared with a target value obtained from the training data to determine an error. The error is determined based on the difference between the target value and the output of operation 302

$$( e . g . , error = \frac{1}{2} {( target value - output of operation 302 )}^{2} ) ,$$

and then the error is used in backpropagation to adjust all the weights of the ANN in operation 303. In operation 303, backward propagation (i.e., backpropagation) of the error is performed on the ANN to adjust all of the weights of the ANN based on the error and the contribution of each weight to the error in order to decrease the error. Operation 303 can be performed, for example, using the delta rule, which is an example of a backpropagation algorithm. The delta rule is a gradient descent learning rule for updating the weights of the inputs to nodes in an ANN. Using a differentiable function, such as a sigmoid or hyperbolic tangent function, for the transfer function ƒ(s) in the nodes can help to decrease the error during backpropagation.

Then, in decision operation 304, a determination is made as to whether another sample in the training data can be used to further train the ANN. If the training data includes an additional sample that has not yet been used to train the ANN, then the operations 302 and 303 are repeated using this additional sample in the training data. After decision operation 304, operations 302 and 303 are repeated for each additional sample in the training data that has not yet been used to train the ANN, until operations 302-303 have been performed for each sample in the training data. If a determination is made at decision operation 304 that each of the samples in the training data has been used to train the ANN in operations 302-303, then the process of FIG. 3 ends. The training of the ANN is then complete. The ANN can be re-trained at any time using a different set of training data having labels or classifications. In addition, the number of input nodes and the number of hidden nodes in the ANN can be changed at any time.

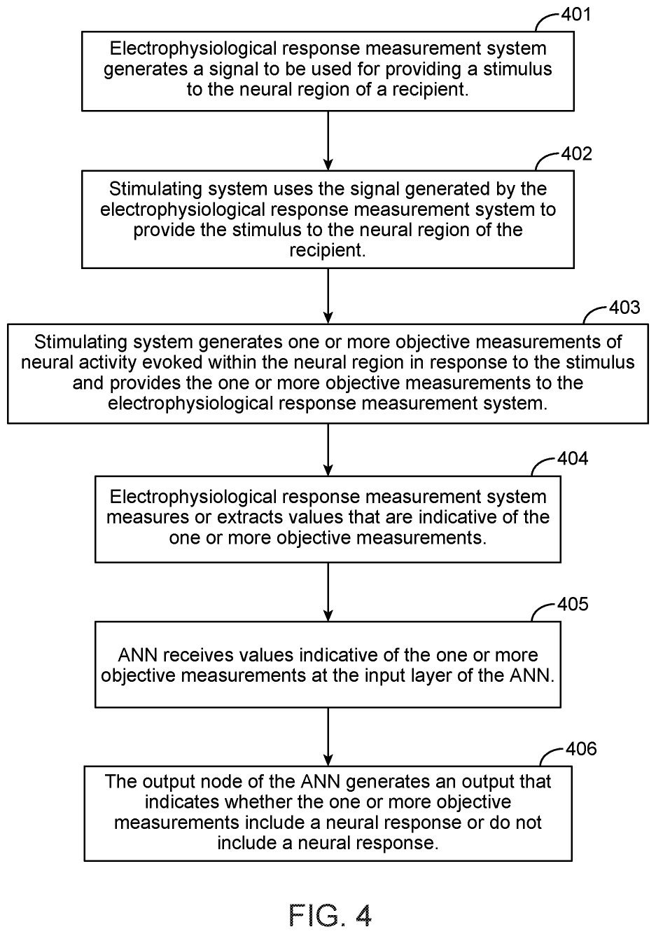

FIG. 4 depicts a flow chart that illustrates examples of operations that can be performed to determine if a measurement of neural activity performed after applying a stimulus to a neural region of a recipient includes a neural response using an artificial neural network (ANN) that has been trained according to the operations of FIG. 3. In operation 401, an electrophysiological response measurement system (e.g., electrophysiological response measurement system 160 of FIG. 1B) generates a signal to be used for providing a stimulus to a neural region (e.g., the auditory nerve) of a recipient. In operation 402, a stimulating system, such as a cochlear implant system, then uses the signal generated by the electrophysiological response measurement system to provide the stimulus (e.g., an electrical stimulus) to the neural region of the recipient (e.g., using an electrode in an electrode array). In operation 403, the stimulating system generates one or more objective measurements of neural activity evoked within the neural region in response to the stimulus, and the stimulating system then provides the one or more objective measurements to the electrophysiological response measurement system (e.g., as one or more signals). In an embodiment, the stimulating system is a cochlear implant system (e.g., as shown in FIGS. 1A-1B) having an implantable component with an array of electrodes implanted in a recipient's cochlea, and the cochlear implant system stimulates each of the electrodes in the array in response to corresponding signals generated by, and received from, the electrophysiological response measurement system during multiple iterations of operations 401-406.

The electrophysiological response measurement system then receives the one or more objective measurements from the stimulating system (e.g., as one or more signals). In operation 404, the electrophysiological response measurement system measures or extracts values that are indicative of the one or more objective measurements, for example, from the one or more signals received from the stimulating system. The values indicative of the one or more objective measurements can, for example, be displayed as a signal trace on a display screen. The electrophysiological response measurement system includes an ANN that has been trained according to the operations of FIG. 3 disclosed herein. The electrophysiological response measurement system provides the values that are indicative of the one or more objective measurements to the input layer of the ANN. In operation 405, the ANN receives the values that are indicative of the one or more objective measurements at the input layer of the ANN. In operation 406, the output node of the ANN generates an output that indicates whether the one or more objective measurements include a neural response to the stimulus or do not include a neural response to the stimulus. The one or more objective measurements may, for example, include noise that is not indicative of a neural response of the auditory nerve to the stimulus.

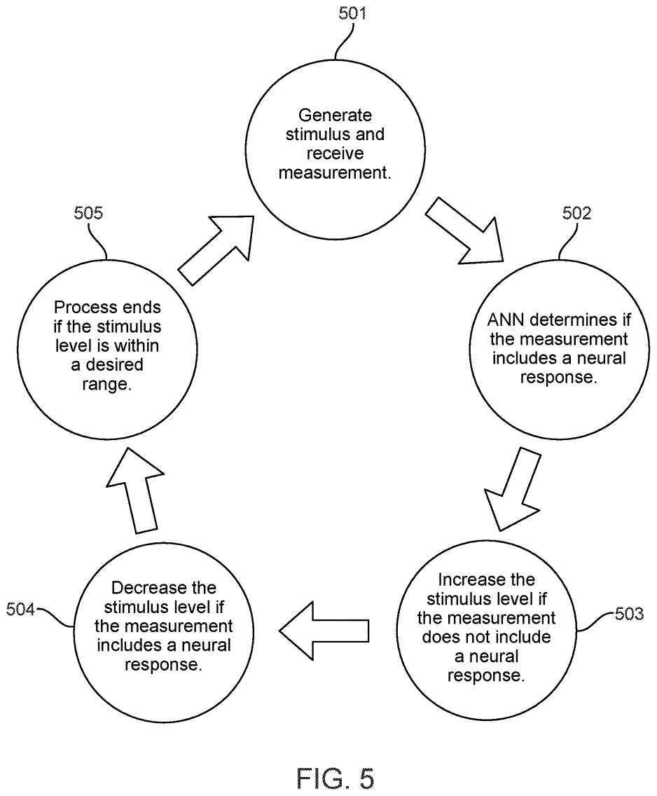

FIG. 5 depicts a flow chart that illustrates examples of operations 501-505 that can be performed to determine a stimulus level that evokes a neural response from a neural region of a recipient within a search range of stimuli levels. The operations 501-505 of FIG. 5 are performed using an artificial neural network (ANN) that has been trained according to the operations of FIG. 3. The operations of FIG. 5 implement a binary search algorithm for a stimulus level that is within the search range of stimuli levels. The operations 501-505 of FIG. 5 can, for example, be performed by a computer system in an electrophysiological response measurement system. The computer system implements the ANN. The operations 501-505 of FIG. 5 can, for example, be performed for each stimulating contact (e.g., each electrode) that is implanted in a recipient's cochlea in an implantable component of a cochlear implant system, such as the cochlear implant system 100 of FIGS. 1A-1B disclosed herein.

In operations 501, the electrophysiological response measurement system and the stimulating system generate a stimulus to the neural region (e.g., the auditory nerve) of the recipient, receive one or more measurements of neural activity evoked in the neural region in response to the stimulus, and provide values that are indicative of the one or more measurements to the input layer of the ANN. Operations 501 can, for example, generate one or more stimuli at one or more stimulating contacts (e.g., one or more electrodes) implanted in a recipient's cochlea in a cochlear implant system. Operations 501 can, for example, generate one or more objective measurements of neural activity evoked within the neural region at one or more of the stimulating contacts (e.g., one or more of the electrodes) in the cochlear implant system. Operations 501 can include the operations 401-405 disclosed herein with respect to FIG. 4. The level of the initial stimulus provided in operations 501 can be selected to be, for example, in the middle of a search range of stimuli levels that range from an expected minimum threshold level (T level) to an expected maximum comfort level (C level) for one or more stimulation channels or electrodes in the stimulating system.

In operation 502, the ANN determines if each measurement received in operations 501 includes a neural response to the stimulus. In operation 502, the ANN outputs a value that indicates whether the measurement includes a neural response or does not include a neural response of the neural region to the stimulus (e.g., merely indicative of noise). Operation 502 can include operation 406 disclosed herein with respect to FIG. 4. In operation 503, the electrophysiological response measurement system selects an increased stimulus level to be provided to the neural region of the recipient if the measurement analyzed by the ANN in operation 502 is determined not to include a neural response. The electrophysiological response measurement system can select an increased stimulus level in operation 503 that is, for example, halfway between the stimulus level previously provided in operations 501 and the maximum stimulus level of the search range of stimuli levels.

In operation 504, the electrophysiological response measurement system selects a decreased stimulus level to be provided to the neural region of the recipient if the measurement analyzed by the ANN in operation 502 is determined to include a neural response. The electrophysiological response measurement system can select a decreased stimulus level in operation 504 that is, for example, halfway between the stimulus level previously provided in operations 501 and the minimum stimulus level of the search range of stimuli levels.

In operation 505, the process of FIG. 5 terminates if the stimulus level selected in the previous iteration of operation 503 or 504 is at a desired stimulus level or within a desired range of stimuli levels. As an example, the process of FIG. 5 can be terminated in operation 505 if the stimulus level selected in the previous iteration of operation 503 or 504 is determined to be equal to, or close to, the lowest level of stimulus that evokes a neural response of the auditory nerve, such as the threshold of the neural response.

The process of FIG. 5 proceeds back to operations 501 if the stimulus level selected in the previous iteration of operation 503 or 504 is not determined to be at a desired stimulus level or within the desired range of stimuli levels in operation 505. Operations 501 are then repeated. In the second and subsequent iterations of operations 501, the electrophysiological response measurement system and the stimulating system generate a stimulus to the neural region of the recipient at the increased or decreased stimulus level selected in the previous iteration of operation 503 or 504. A measurement of neural activity evoked in response to the stimulus is then received, and values indicative of the measurement are provided to the input layer of the ANN. Operations 502-505 are then repeated following each iteration of operations 501, until the process of FIG. 5 terminates as described above.

The operations 501-505 of FIG. 5 can be performed, for example, for each stimulating contact (e.g., for each electrode) implanted in a recipient's cochlea in a cochlear implant system. As a specific example that is not intended to be limiting, the operations 501-505 of FIG. 5 can be performed to determine a neural response for each of the electrodes in a cochlear implant system, such as the stimulating contacts 126 of FIGS. 1A-1B. After a neural response is determined for one electrode, operations 501-505 can be performed for another electrode in the electrode array of the cochlear implant system. The electrodes in the electrode array can be stimulated in any desired order during iterations of operations 501. The neural response determined in operations 501-505 for each of the electrodes in a cochlear implant system can, for example, be used to generate the dynamic range, including the minimum threshold level (T level) and/or the maximum comfort level (C level), for stimulation of each of the electrodes.

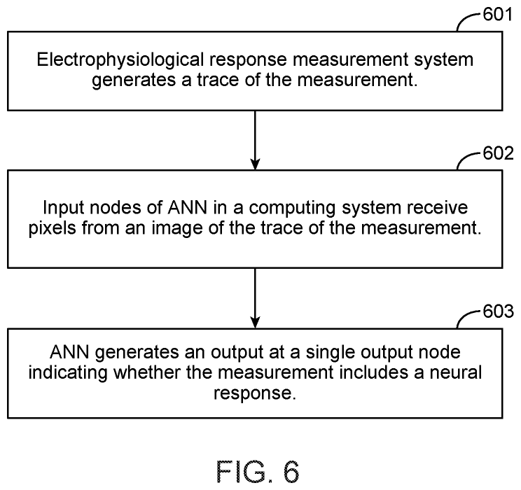

FIG. 6 depicts a flow chart that illustrates examples of operations that can be performed to determine if a measurement performed after applying a stimulus to a neural region of a recipient includes a neural response by providing pixels of a trace of the measurement to an artificial neural network (ANN). The ANN used in the operations of FIG. 6 can be trained according to the operations of FIG. 3. Initially, the operations 401-403 of FIG. 4 are performed to provide a stimulus to the neural region (e.g., auditory nerve) of the recipient, generate a measurement of neural activity evoked in the neural region in response to the stimulus, and provide the measurement to an electrophysiological response measurement system.

In operation 601, the electrophysiological response measurement system generates a trace of the measurement. The electrophysiological response measurement system generates an image of the trace that is formed of pixels. As a specific example that is not intended to be limiting, the electrophysiological response measurement system can generate an N×N image of the trace that is formed of N² pixels, where N is any positive integer greater than 0. The electrophysiological response measurement system provides the pixels from the image of the trace to the ANN. The ANN includes an input layer that has input nodes (e.g., an N² number of input nodes), for example, as shown in FIG. 2. The ANN is executed by a computing system.

In operation 602, the input nodes in the input layer of the ANN receive the pixels from the image of the trace of the measurement. Each of the input nodes in the ANN receives a different/unique one of the pixels from the image of the trace. For example, each of N² input nodes in the ANN can receive a different one of N² pixels from the image of the trace. In operation 603, the ANN generates an output at a single output node of the ANN that indicates whether the measurement includes a neural response (e.g., an output of 1) or does not include a neural response (e.g., an output of 0). The ANN can include one or more hidden layers between the input layer and the output layer, as disclosed herein, for example, with respect to FIG. 2.

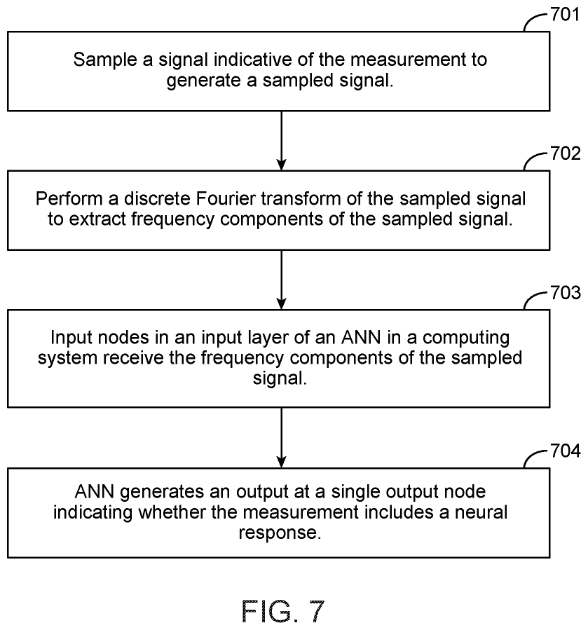

FIG. 7 depicts a flow chart that illustrates examples of operations that can be performed to determine if a measurement performed after applying a stimulus to a neural region of a recipient includes a neural response by providing frequency components of a sample of the measurement to an artificial neural network (ANN). The ANN used in the operations of FIG. 7 can be trained according to the operations of FIG. 3. Initially, the operations 401-403 of FIG. 4 are performed to provide a stimulus to the neural region (e.g., auditory nerve) of the recipient, generate a measurement of neural activity evoked in the neural region in response to the stimulus, and provide the measurement to an electrophysiological response measurement system.

In operation 701, a signal that is indicative of the measurement is sampled to generate a sampled signal (e.g., using a sampler in the electrophysiological response measurement system). The signal indicative of the measurement sampled in operation 701 can, for example, be a waveform from a trace that is assumed to be a finite-duration signal representing one period of a repeating, periodic signal. In operation 702, a discrete Fourier transform (DFT) of the sampled signal is performed (e.g., using a fast Fourier transform) to extract frequency components of the sampled signal. Each of the frequency components of the sampled signal represents one frequency of the sampled signal. Operation 702 can, for example, be performed by software in the electrophysiological response measurement system. The frequency components of the sampled signal (or a subset of the frequency components of the sampled signal) are then provided to an input layer of the ANN. The ANN is executed by a computing system.

In operation 703, the input nodes in the input layer of the ANN receive the frequency components of the sampled signal (or a subset of the frequency components). Each of the input nodes in the ANN receives a different/unique one of the frequency components from the sampled signal. Thus, an N number of the frequency components of the sampled signal are received by an N number of the input nodes of the ANN. In operation 704, the ANN generates an output at a single output node of the ANN that indicates whether the measurement includes a neural response (e.g., generates an output of 1) or does not include a neural response (e.g., generates an output of 0). The ANN can include one or more hidden layers between the input layer and the output layer, as disclosed herein, for example, with respect to FIG. 2.

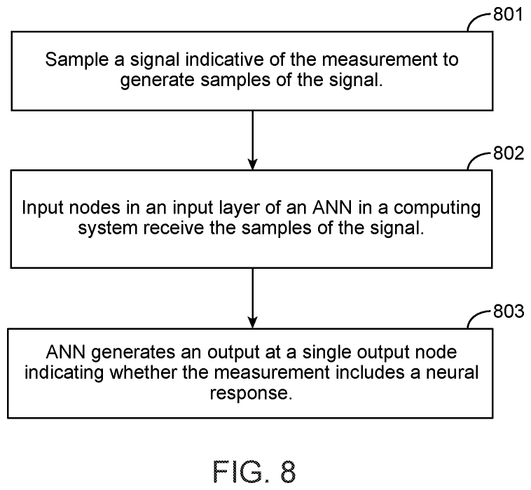

FIG. 8 depicts a flow chart that illustrates examples of operations that can be performed to determine if a measurement performed after applying a stimulus to a neural region of a recipient includes a neural response by providing samples of a signal indicative of the measurement to an artificial neural network (ANN). The ANN used in the operations of FIG. 8 can be trained according to the operations of FIG. 3. Initially, the operations 401-403 of FIG. 4 are performed to provide a stimulus to the neural region (e.g., auditory nerve) of the recipient, generate a measurement of neural activity evoked in the neural region in response to the stimulus, and provide the measurement to an electrophysiological response measurement system.

In operation 801, a signal that is indicative of the measurement is sampled to generate samples of the signal (e.g., using a sampler in the electrophysiological response measurement system). The signal indicative of the measurement sampled in operation 801 can, for example, be a waveform from a trace that is assumed to be a finite-duration signal representing one period of a repeating, periodic signal. The samples of the signal (or a subset of the samples) are then provided to an input layer of the ANN. The ANN is executed by a computing system.

In operation 802, the input nodes in the input layer of the ANN receive the samples of the signal (or a subset of the samples). Each of the input nodes in the ANN receives a different/unique one of the samples of the signal. Thus, an N number of the samples of the signal are received by an N number of the input nodes of the ANN. In operation 803, the ANN generates an output at a single output node of the ANN that indicates whether the measurement includes a neural response (e.g., generates an output of 1) or does not include a neural response (e.g., generates an output of 0). The ANN can include one or more hidden layers between the input layer and the output layer, as disclosed herein, for example, with respect to FIG. 2.

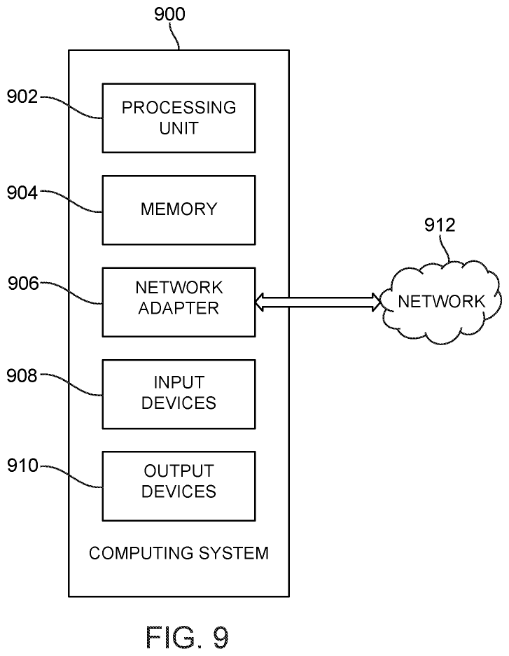

FIG. 9 illustrates an example of a suitable computing system 900 that can perform any of the operations or functions disclosed herein. For example, computing system 900 can be used to implement any of the ANNs disclosed herein. Computing system 900 can generate an indication of whether a measurement of neural activity evoked in a neural region in response to stimulus includes a neural response or does not include a neural response using an ANN, as disclosed herein. Computing system 900 can, for example, be part of an electrophysiological response measurement system. Computing systems, environments, or configurations that can be suitable for use with examples disclosed herein include, but are not limited to, personal computers, server computers, hand-held devices, laptop devices, multiprocessor systems, microprocessor-based systems, programmable consumer electronics (e.g., smart phones), network computers, minicomputers, mainframe computers, tablets, distributed computing environments that include any of the above systems or devices, and the like. The computing system 900 can be a single virtual or physical device operating in a networked environment over communication links to one or more remote devices. The remote device can be an auditory prosthesis (e.g., the auditory prosthesis of FIGS. 1A-1B), a personal computer, a server, a router, a network personal computer, a peer device or other common network node.

Computing system 900 includes at least one processing unit 902 and memory 904. The processing unit 902 includes one or more hardware or software processors (e.g., Central Processing Units) that can obtain and execute instructions. The processing unit 902 can communicate with and control the performance of other components of the computing system 900. The memory 904 is one or more software-based or hardware-based computer-readable storage media operable to store information accessible by the processing unit 902.

The memory 904 can store instructions executable by the processing unit 902 to implement applications (software) or cause performance of any of the functions or operations disclosed herein, as well as store other data. The memory 904 can be volatile memory (e.g., random access memory or RAM), non-volatile memory (e.g., read-only memory or ROM), or combinations thereof. The memory 904 can also include one or more removable or non-removable storage devices. The memory 904 can include transitory memory and/or non-transitory computer-readable storage media. Non-transitory computer-readable storage media is tangible computer-readable storage media that stores data for access at a later time, as opposed to media that only transmits propagating electrical signals, such as wires. In examples, the memory 904 can include non-transitory computer-readable storage media, such as RAM, ROM, EEPROM (Electronically-Erasable Programmable Read-Only Memory), flash memory, optical disc storage, magnetic storage, solid state storage, or any other memory media usable to store information for later access. In examples, the memory 904 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 904 can include wired media such as a wired network or direct-wired connection, and wireless media such as acoustic, radio-frequency, infrared and other wireless media or combinations thereof.

In the illustrated example, the computing system 900 further includes a network adapter 906, one or more input devices 908, and one or more output devices 910. The system 900 can include other components, such as a system bus, component interfaces, a graphics system, a power source (e.g., a battery), among other components.

The network adapter 906 is a component of the computing system 900 that provides network access to network 912. The network adapter 906 can provide wired or wireless network access and can support one or more of a variety of communication technologies and protocols, such as Ethernet, cellular, Bluetooth, near-field communication, and RF (Radio-frequency), among others. The network adapter 906 can include one or more antennas and associated components configured for wireless communication according to one or more wireless communication technologies and protocols.

The one or more input devices 908 are devices over which the computing system 900 receives input from a user. The one or more input devices 908 can include physically-actuatable user-interface elements (e.g., buttons, switches, or dials), touch screens, keyboards, mice, pens, and voice input devices, among others input devices.

The one or more output devices 910 are devices by which the computing system 900 is able to provide output to a user. The output devices 910 can include displays, speakers, and printers, among other output devices.

Any embodiment or any feature disclosed herein can be combined with any one or more other embodiments and/or other features disclosed herein, unless explicitly indicated otherwise. Any embodiment or any feature disclosed herein can be explicitly excluded from use with any one or more other embodiments and/or other features disclosed herein, unless explicitly indicated otherwise. It is noted that any method detailed herein also corresponds to a disclosure of a device and/or system configured to execute one or more or all of the method actions associated with the device and/or system as detailed herein. It is further noted that any disclosure of a device and/or system detailed herein corresponds to a method of making and/or using that device and/or system, including a method of using that device according to the functionality detailed herein.

The foregoing description of the exemplary embodiments of the present invention has been presented for the purpose of illustration. The foregoing description is not intended to be exhaustive or to limit the present invention to the examples disclosed herein. In some instances, features of the present invention can be employed without a corresponding use of other features as set forth. Many modifications, substitutions, and variations are possible in light of the above teachings, without departing from the scope of the present invention.

1. A computing system comprising:
at least one processing unit that implements an artificial neural network, wherein the artificial neural network generates an output at a single output node that indicates whether a measurement performed after a stimulus to a neural region of an individual includes a neural response.

2. The computing system of claim 1, wherein input nodes of the artificial neural network receive pixels from an image of a trace of the measurement.

3. The computing system of claim 1, wherein input nodes of the artificial neural network receive frequency components of a sampled signal generated by sampling a signal indicative of the measurement.

4. The computing system of claim 1, wherein input nodes of the artificial neural network receive samples of a signal indicative of the measurement.

5. The computing system of claim 1, wherein the computing system trains the artificial neural network using training data that comprises measurements performed after stimuli to the neural region and labels or classifications that indicate whether the measurements include neural responses.

6. The computing system of claim 1, wherein an electrophysiological response measurement system generates a trace indicative of the measurement and provides values indicative of the trace to an input layer of the artificial neural network.

7. The computing system of claim 1, wherein an electrophysiological response measurement system performs a search algorithm by generating stimuli at different levels to be applied to the neural region and analyzes measurements from the individual to determine a stimulus level that generates the neural response from the neural region.

8. The computing system of claim 1, wherein a stimulating system provides stimuli to the neural region using stimulating contacts, wherein a set of values indicative of an objective measurement after each of the stimuli is provided to input nodes of the artificial neural network, and wherein the single output node generates an additional output indicating whether each of the objective measurements includes a neural response.

9. The computing system of claim 1, wherein the single output node is the only output node of the artificial neural network that generates the output that indicates whether the measurement includes a neural response.

10. A method comprising:
receiving, at an artificial neural network in a computing system, values indicative of a measurement performed after a stimulus provided to a neural region of an individual; and
generating an output that indicates whether the measurement comprises a neural response at a single output node of the artificial neural network.

11. The method of claim 10 further comprising:
generating the values indicative of the measurement using an electrophysiological response measurement system.

12. The method of claim 10, wherein receiving the values indicative of the measurement further comprises:
receiving, at input nodes of the artificial neural network, pixels from an image of a trace of the measurement.

13. The method of claim 10, wherein receiving the values indicative of the measurement further comprises:
receiving, at input nodes of the artificial neural network, frequency components of a sampled signal generated by sampling a signal indicative of the measurement.

14. The method of claim 10, wherein receiving the values indicative of the measurement further comprises:
receiving, at input nodes of the artificial neural network, samples of a signal indicative of the measurement.

15. The method of claim 10 further comprising:
providing the stimulus to a first stimulating contact in a stimulating system;
receiving the measurement at a second stimulating contact in the stimulating system; and
providing the measurement to the computing system.

16. The method of claim 10 further comprising:
increasing a stimulus level provided to the neural region if the output indicates that the measurement does not comprise a neural response; and
decreasing the stimulus level provided to the neural region if the output indicates that the measurement comprises a neural response.

17. The method of claim 10, wherein generating the output that indicates whether the measurement comprises a neural response further comprises:
generating the output that indicates whether the measurement comprises a neural response at only one output node of the artificial neural network.

18. A non-transitory computer-readable storage medium comprising computer-readable instructions stored thereon for causing a computing system to:
receive, at input nodes of an artificial neural network in the computing system, pixels from an image of a trace of a measurement performed after a stimulus to an auditory nerve of an individual; and
generate an indication of whether the measurement comprises a neural response based on the pixels using the artificial neural network.

19. The non-transitory computer-readable storage medium of claim 18, wherein the computer-readable instructions further cause the computing system to:
receive a different one of the pixels at each of the input nodes of the artificial neural network.

20. The non-transitory computer-readable storage medium of claim 18, wherein the computer-readable instructions further cause the computing system to:
generate the indication of whether the measurement comprises a neural response or does not comprise a neural response at a single output node of the artificial neural network.

21. (canceled)

22. (canceled)

23. (canceled)

24. (canceled)

25. (canceled)
